(19) United States
(12) Patent Application Publication
(10) Pub. No.: US 2007/0088088 A1
(43) Pub. Date: Apr. 19, 2007

(54) Method for preventing or treating metabolic syndrome

(75) Inventors: Yoichi Inada, Azumino-shi (JP); Shigeru Nakano, Azumino-shi (JP); Hiroaki Masuzaki, Kyoto-shi (JP)

Correspondence Address: SUGHRUE-265550, 2100 PENNSYLVANIA AVE. NW, WASHINGTON, DC, 20037-3213, US

(73) Assignee: KISSEI PHARMACEUTICAL CO., LTD.

(21) Appl. No.: 11/406,339

(22) Filed: Apr. 19, 2006

(30) Foreign Application Priority Data
Oct. 18, 2005 (JP) P.2005-303264
Jan. 19, 2006 (JP) P.2006-010882

(51) Int. Cl. A61K 31/192

(52) U.S. Cl. 514/571

(57) ABSTRACT

A method for preventing or treating metabolic syndrome by administering bezafibrate. Since bezafibrate suppresses the action of 11β-hydroxysteroid dehydrogenase type 1 and also accelerates expression of adiponectin receptor, it is used as an agent for preventing or treating metabolic syndrome.

### FIELD OF THE INVENTION

This invention relates to a method for preventing or treating metabolic syndrome. More specifically, the invention relates to a method for preventing or treating metabolic syndrome by inhibiting overexpression of 11β-hydroxysteroid dehydrogenase type 1 and accelerating expression of adiponectin receptor, through the administration of bezafibrate.

### BACKGROUND OF THE INVENTION

Metabolic syndrome is a disease with complications such as risk factors of high triglyceride, low HDL-cholesterolemia, abnormal glucose metabolism and hypertension, with the background of accumulated visceral fat. Even when the individual symptoms are not severe, the onset of these complications involves a high-risk of the occurrence of arteriosclerotic diseases, so that patients with metabolic syndrome draw attention as a high-risk group of arteriosclerotic diseases. WHO defines that an individual with at least one symptom of type 2 diabetes mellitus, abnormal glucose tolerance and insulin resistance and at least two symptoms of hypertension, obesity, abnormal lipid metabolism (high triglyceridemia, low HDL-cholesterolemia) and microalbuminurea is a patient with metabolism syndrome.

Further, NCEP ATP III (National Cholesterol Education Program: Adult Treatment Panel III, 16, 2001) defines that an individual with three or more of the following criteria should be diagnosed as metabolic syndrome.

TABLE 1
Diagnostic criteria of metabolic syndrome
NCEP ATP-III

| Risk factor | Diagnostic criteria | |
|---|---|---|
| Abdominal obesity | (abdominal length) | |
| Male | >102 | cm |
| Female | >88 | cm |
| Triglyceride | ≧150 | mg/dl |
| HDL-cholesterol | | |
| Male | <40 | mg/dl |
| Female | <50 | mg/dl |
| Blood pressure | ≧130/85 | mmHg |
| Blood glucose during fasting | ≧110 | mg/dl |

For therapeutic treatment of metabolic syndrome, attempts are currently made for drug therapies of the individual risk factors. That is, fibrate or statin derivatives are used for abnormal lipid metabolism; sulfonyl urea derivatives, α-glucosidase inhibitors or insulin sensitizer agents are used for abnormal glucose metabolism; and angiotensin converting enzyme inhibitors or adrenalin α receptor antagonistic agents are used for hypertension. However, obesity with visceral fat accumulation as the background disease of metabolic syndrome is mainly treated by exercise therapy and dietary therapy, and only central appetite suppressors are used as pharmaceutical therapy.

Accordingly, a pharmaceutical agent which shows its effect on all of the abnormal lipid metabolism, abnormal glucose metabolism, hypertension and the related risk factors and is also effective upon obesity with visceral fat accumulation as the background disease is most desirable for the treatment of metabolic syndrome. However, to date no information is available concerning a pharmaceutical agent which shows such an effect by its single use.

Bezafibrate is broadly used as a hyperlipemia treating agent and is known to be effective for high triglyceride, low HDL-cholesterolemia or the related abnormal lipid metabolism. It is considered that its serum triglyceride lowering action mechanism is acceleration of triglyceride catabolism via the activation of PPARα which is one of the subtypes of peroxisome proliferator-activated receptor (to be referred to as “PPAR” hereinafter). In addition, it is known that bezafibrate is also effective for abnormal glucose metabolism through the improvement of insulin resistance by increase of plasma adiponectin concentration (Mori et al., Endocrine, vol. 25, pp. 247-251, 2004). Further, It is known also that bezafibrate shows a hypotensive action and is therefore effective for hypertension (Hypertens. Res., 26, 307-313, 2003). However, it is not known so far that bezafibrate has the action to improve obesity with visceral fat accumulation directly without any lipid metabolism improving action.

Adiponectin is a species of a physiologically active substance adipocytokine which is secreted from adipose tissue, and is known as a protective factor concerned in the insulin resistance, fibrosis of the liver, malignant tumor and the like and also for arteriosclerosis. For example, it has been reported that blood adiponectin level is reduced in patients of diabetes and obesity, it takes an important role as a protective factor for fibrosis of the liver in the case of hepatitis, and in the case of metabolic syndrome, the blood adiponectin level is reduced in inverse proportion to the degree of obesity, showing reverse-correlation with the insulin resistance index. In addition, this is also drawing attention as a target molecule of PPARγ in the adipose tissue of obesity patients accompanying insulin resistance (H. Maeda et al., Adiposcience, vol. 1, pp. 247-257, 2004). It is considered that the action of bezafibrate to increase plasma adiponectin level is based on the agonistic action of bezafibrate upon adiponectin receptor, but when use of the agonist is continued for a prolonged period of time, reduction of sensitivity for the agonist is frequently observed. It is considered that this phenomenon occurs because of the generation of resistance such as reduction of receptor expression level in a target organ due to a feed back mechanism in the living body. Accordingly, a pharmaceutical agent which can increase sensitivity for adiponectin and thereby keep its action further prolonged period of time, by increasing expression level of adiponectin receptor, is desirable.

The adiponectin receptor (to be referred sometimes to as “AdipoR” hereinafter) is distributed in the liver and skeletal muscle which are important in regulating insulin sensitivity and concerned in the incorporation of glucose into tissues and β-oxidation of fatty acids via PPARα (T. Yamauchi et al., Molecular Medicine, A Special Issue, Frontier of Life Style-related Diseases (written in Japanese), no. 42, pp. 125-133).

The AdipoR exists in two species of subtypes AdipoR1 and AdipoR2 (Yamauchi et al., Nature, vol. 423, pp. 762-769, 2003), and AdipoR1 is distributed in the whole body organ and AdipoR2 is mainly distributed in the liver. It has been reported that stimulation of AdipoR1 in the liver accelerates activation of AMP kinase (inhibition of gluconeogenesis) and stimulation of AdipoR2 accelerates β-oxidation of fatty acids via the activation of PPARα (M. Kobayashi et al., The Japanese Journal of Obesity (written in Japanese), vol. 11 (Supplement), p. 152, 2005).

In addition, a relationship between a ligand responsive transcription factor, PPAR, and obesity is also drawing attention (Masuzaki H et al., Current Drug Targets—Immune, Endocrine & Metabolic Disorders, vol. 3, pp. 255-262, 2003). PPAR belongs to a nuclear receptor family which uses glucocorticoid, androgen, progesterone, mineral corticoid, estrogen, activated vitamin D and the like as ligands, and PPARα, γ and δ subtypes exist therein.

PPARα is expressed in the liver, myocardial muscle, digestive tract, vascular endothelial cell, aorta smooth muscle cell, macrophage, lymphocyte and the like and is involved in lipid catabolism such as the acceleration of the β-oxidation of fatty acid and the activation of lipoprotein lipase (LPL) in the liver.

PPARγ is mainly expressed in adipose tissue and is involved in lipid anabolism such as the differentiation of adipose tissue-and the promotion of lipid synthesis in adipose tissue.

PPARδ is expressed in many tissues including skeletal muscle and brown adipose tissue, and is involved in the activation of the oxidation of fatty acid.

By some approaches for defining metabolic syndrome as the abnormality of adipose tissue function, an abnormal activation mechanism of glucocorticoid action in adipose tissue is elucidated. The activity of an intracellular glucocorticoid reactivating enzyme, namely 11β-hydroxysteroid dehydrogenase type 1 (to be referred to as “11β-HSD1” hereinafter), in adipose tissue increases in a manner depending on the obesity level and has a good correlation with insulin resistance index. In addition, the activity of the enzyme 11β-HSD1 and the gene expression level thereof in adipose tissue are significantly suppressed by insulin sensitizers typically including PPARγ agonists such as thiazolidinedione (TZD) derivatives as therapeutic agents for diabetes. Accordingly, the meaning of 11β-HSD1 as a target molecule of PPARγ in adipose tissue is now drawing attention (see Masuzaki H., et al., Current Drug Targets—Immune, Endocrine & Metabolic Disorders, 2003, Vol. 3, p. 255-262).

It is known that a transgenic mice (aP2HSD1 mice) excessively expressing 11β-HSD1 involve the increase of the enzyme activity, which corresponds to obesity, and also exerts main elements of metabolic syndrome. In the mice, 11β-HSD1 increase at about the same level as in genetic obesity ob/ob mice or persons with severe obesity, additionally involving about 15% body weight increase compared with control mice loaded with normal diet and also involving a prominent increase of the weight of the mesenteric adipose tissue in particular among adipose tissues (Masuzaki H., et al., Science, 2001, Vol. 294, pp. 2166-2170).

On the other hand, a 11β-HSD1 knockout mice exert apparent resistance against onset of diabetes without causing induction of hepatic gluconeogenesis enzymes by loaded stress and high-fat diet, and in this mouse, expressions of a group of molecules related to lipid anabolism and transcription factors which control their expression are markedly increased in the liver. In addition, it is known that the accumulation of visceral fat tissue and the occurrence of metabolic abnormality as derived from high-fat diet and the mating with ob/ob mice are suppressed, so that the knockout mice are hardly afflicted with metabolic syndrome (Kotelevtsev Y. et al., Proc. Natl. Acad. Sci. USA, 2004, vol. 94, pp. 14924-14929).

Thus, 11β-HSD1 is one of the main factors of the onset of metabolic syndrome, and a pharmaceutical agent suppressing the action can be used as a prophylactic or therapeutic agent of metabolic syndrome.

Meanwhile, the action mechanisms of bezafibrate and other fibrates are diverse, such pharmaceutical agents have individually inherent characteristic properties, and the actions of the individual agents as ligands toward PPARα are common. However, none of the action of the fibrates toward 11β-HSD1, particularly the action thereof toward tissue 11β-HSD1 has been known.

By the way, a recent report describes that bezafibrate suppresses the onset of myocardial infarction in patients with metabolic syndrome (Alexander Tenenbaum, et al., Arch Intern Med., 2005, Vol. 165, p. 1154-1160). However, the report just concerns a symptomatic therapy of one symptom of complicated metabolic syndrome, and it never tells about the radical therapy of metabolic syndrome by therapeutically treating obesity with visceral fat accumulation as one of the background diseases.

### SUMMARY OF THE INVENTION

It is an object of the invention to provide a method for suppressing the expression of 11β-HSD1, which can be used for the prevention or treatment of metabolic syndrome and to provide a method for preventing or treating metabolic syndrome using the same.

The present inventors have conducted extensive studies so as to meet the object and, as a result, found that bezafibrate shows excellent activity for suppressing expression of 11β-HSD1, particularly shows the 11β-HSD1 expression suppressing activity in mesenteric adipose tissue, and has an activity of accelerating expression of adiponectin receptor, thereby accomplishing the invention.

That is, the gist of the invention resides in a method for preventing or treating metabolic syndrome or obesity by administering bezafibrate and a method for suppressing expression of 11β-HSD1 by administering bezafibrate.

Bezafibrate shows effects on any of the high triglyceride, low HDL-cholesterolemia, abnormal glucose metabolism and hypertension and also has excellent activities of the suppression of the expression of 11β-HSD1, particularly 11β-HSD1 expression suppression activity in mesenteric adipose tissue, so that it can be used for the prevention or treatment of metabolic syndrome, particularly metabolic syndrome which accompanies obesity with visceral fat accumulation and for the prevention or treatment of obesity, particularly obesity with visceral fat accumulation.

### BRIEF DESCRIPTION OF THE DRAWINGS

By way of example and to make the description more clear, reference is made to the accompanying drawing in which:

FIG. 1 is a graph showing AdipoR1 and AdipoR2 mRNA expression levels in cells at 24 hours after the addition of bezafibrate and fenofibric acid, an active metabolite of fenofibrate, to murine hepatoma Hepa1-6 cells. From the left, bar graphs represent AdipoR1 and AdipoR2 mRNA expression levels of Control: control group; BF 100: bezafibrate 100 μmol/l addition group; BF 300: bezafibrate 300 μmol/l addition group; FEN 100: fenofibric acid 100 μmol/l addition group; and FEN 300: fenofibric acid 100 μmol/l addition group, respectively. The axis of ordinate shows mean value (%) and standard error of the mean (SEM) of expression levels in respective groups, wherein the mRNA expression level of the control group is defined as 100%. In the drawing, the marks * and ** show significant differences versus control group at less than 5% and 1%, respectively.

FIG. 2 is a graph showing AdipoR1 and AdipoR2 mRNA expression levels in (A) liver and (B) skeletal muscle of db/db mice 8 weeks after the repeated administration of bezafibrate and fenofibrate. From the left, bar graphs represent AdipoR1 and AdipoR2 mRNA expression levels of Control: db/db control mice; BF 100: bezafibrate 100 mg/kg/day administration group; BF 300: bezafibrate 300 mg/kg/day administration group; and FEN 300: fenofibrate 100 mg/kg/day-administration group, respectively. The axis of ordinate shows mean value (%) and SEM of expression levels in respective groups, wherein the mRNA expression levels of db/db control mice is defined as 100%. In the drawing, the marks * and ** show the same meaning as in FIG. 1.

FIG. 3 is a graph showing the effects of the 8 weeks of repeated administration of bezafibrate and fenofibrate to improve diabetes and hyperlipidemia in db/db mice. (A) is a graph showing glycated hemoglobin value (%), (B) is a graph showing plasma glucose concentration (mg/dl), (C) is a graph showing plasma triglyceride concentration (mg/dl) and (D) is a graph showing plasma adiponectin concentration (ng/ml). In each graph, N is normal control mice; C is db/db control mice; and BF 100, BF 300 and FEN 300 are the same as in FIG. 2. Each axis of ordinate shows respective mean value and SEM. In the drawing, the marks # and ## show significant differences between normal control and db/db control mice at significance levels at less than 5% and 1%, respectively, and the marks * and ** show the same meaning as in FIG. 1.

FIG. 4 is a graph showing the effects of bezafibrate and fenofibrate to improve diabetes and hyperlipidemia in db/db mice. From the left, bar graphs represent mean and SEM of normal control mice, db/db control mice, bezafibrate 100 mg/kg/day repeated administration group, bezafibrate 300 mg/kg/day repeated administration group, and fenofibrate 300 mg/kg/day repeated administration group. In the drawing, the mark # shows a significant difference between normal control and db/db control mice (less than 5%). The mark * shows that there is significant difference versus db/db control mice (less than 5%).
(A) is a graph showing glycated hemoglobin value (%) after 8 weeks of repeated administration of bezafibrate or fenofibrate.
(B) is a graph showing plasma glucose concentration (mg/dl) after 8 weeks of repeated administration of bezafibrate or fenofibrate.
(C) is a graph showing plasma triglyceride concentration (mg/dl) after 8 weeks of repeated administration of bezafibrate or fenofibrate.
(D) is a graph showing plasma HDL-cholesterol concentration (mg/dl).

FIG. 5 is a graph showing a result of the determination of 11β-HSD1 mRNA expression levels in respective tissues after repeated administration of bezafibrate and fenofibrate. From the left, bar graphs represent mean and SEM of 11β-HSD1 mRNA expression level in normal control mice, db/db control mice, bezafibrate 100 mg/kg/day repeated administration group, bezafibrate 300 mg/kg/day repeated administration group, and fenofibrate 300 mg/kg/day repeated administration group. The axis of ordinate shows the expression level in each group (%), wherein the mRNA expression level of db/db control mice is defined as 100%. In the drawing, the mark # shows a significant difference between normal control group and db/db control mice (less than 5%). The mark * shows that there is a significant difference versus db/db control mice (less than 5%).
(A) is a graph showing expression of 11β-HSD1 in liver after 8 weeks of repeated administration of bezafibrate or fenofibrate.
(B) is a graph showing expression of 11β-HSD1 in intestinal skeletal muscle tissue after 8 weeks of repeated administration of bezafibrate or fenofibrate.
(C) is a graph showing expression of 11β-HSD1 in mesenteric adipose tissue after 8 weeks of repeated administration of bezafibrate or fenofibrate.
(D) is a graph showing expression of 11β-HSD1 in subcutaneous adipose tissue after 8 weeks of repeated administration of bezafibrate or fenofibrate.

### DETAILED DESCRIPTION OF THE INVENTION

The dosage forms of the prophylactic or therapeutic agent of the invention include oral agents for example powders, granules, tablets, and capsules. These oral agents in the case of tablets for example can be produced by adding necessary fillers, disintegrators, lubricants and the like to the active component, and then tableting the resulting mixture by routine methods. The dose of the active component can be appropriately determined, depending on for example the age and body weight of a patient and the severity of the disease. In the case of bezafibrate, it is administered within a range of generally from 100 to 1,000 mg, preferably from 400 to 600 mg.

### EXAMPLES

The invention is now described in detail in the following Examples and Test Examples. However, the invention is never limited to the contents thereof.

### Example 1

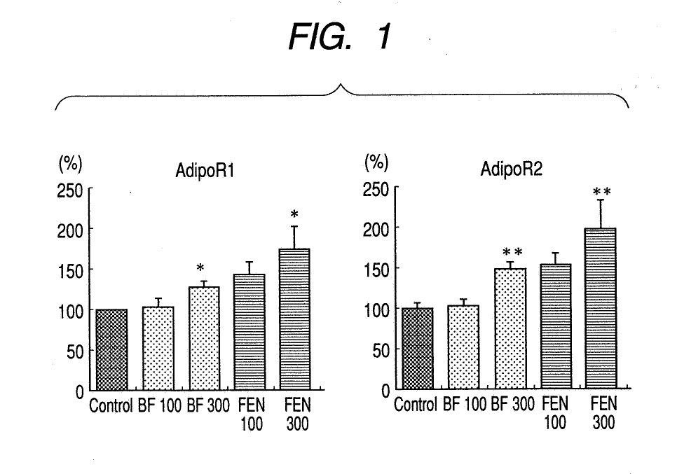

To murine hepatoma Hepa1-6 cells (manufactured by American Type Culture Collection) was added 100 or 300 μmol/l of bezafibrate, 100 or 300 μmol/l of fenofibric acid or a solvent (DMSO (final concentration 1%)) (control group), and 24 hours thereafter, total RNA was purified using SV Total RNA Isolation System™ (manufactured by Promega). Using the thus obtained total RNA as the template, the sample was converted into cDNA by carrying out reverse transcription reaction using ExScript™ RT Reagent Kit (manufactured by Takara Bio), and using this as the template, real time quantitative PCR was carried out by SYBR™ Premix ExTaq™ (manufactured by Takara Bio) using the AdipoR1 primer conventionally known by a reference (Bluer M. et al., Biochem. Biophys. Res. Comm., vol. 329, pp. 1127-1132, 2005) or Perfect Real Time Support System AdipoR2 primer (manufactured by Takara Bio). From this result, amounts of mRNA of AdipoR1 and AdipoR2 in each tissue were calculated. In addition, amount of ribosomal RNA was calculated in the same manner using an internal standard substance Predeveloped TaqMan Assay reagents ribosomal RNA (manufactured by Applied Biosystems Japan), and the ratio with this value (amount of target mRNA/amount of ribosomal RNA) was analyzed as each mRNA expression level. The reaction was carried out using Applied Biosystems GeneAmp 5700 SDS (manufactured by Applied Biosystems Japan). The results are shown in FIG. 1.

In comparison with the control group, bezafibrate significantly increased expression levels of AdipoR1 and AdipoR2 in Hepa1-6 cells.

### Example 2

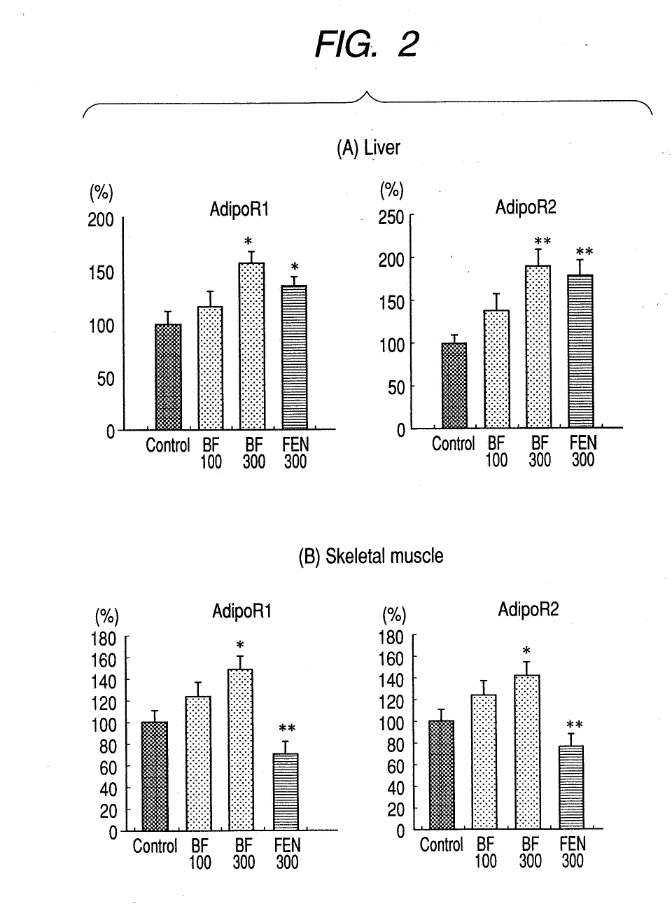

A 1% methyl cellulose solution (control mice), 100 mg/kg or 300 mg/kg of bezafibrate or 300 mg/kg of fenofibrate was orally administered to type 2 diabetic mice, genetic leptin receptor-deficient mice (BKS. Cg-+Leptdb/+Leptdb/Jcl mice; to be referred to as db/db mice hereinafter), repeatedly once a day. After 8 weeks-of the administration, each animal was anesthetized by the intraperitoneal injection of 20% chloral hydrate (manufactured by Wako Pure Chemical Industries) to remove liver and skeletal muscle. Total RNA was extracted from the thus removed tissues using RNA extraction reagent ISOGEN (manufactured by Nippon Gene), and the total RNA was further purified using RNeasy Micro Kit (manufactured by Qiagen). Using the thus obtained RNA as the template, the sample was converted into cDNA by carrying out reverse transcription reaction using ExScript™ RT Reagent Kit (manufactured by Takara Bio). Using this as the template, real time quantitative PCR was carried out by the same method described in Example 1, and expression levels of mRNA of AdipoR1 and AdipoR2 in each tissue was calculated. The results are shown in FIG. 2.

In comparison with db/db control mice, bezafibrate significantly increased expression levels of AdipoR1 and AdipoR2 in the liver and skeletal muscle.

### Example 3

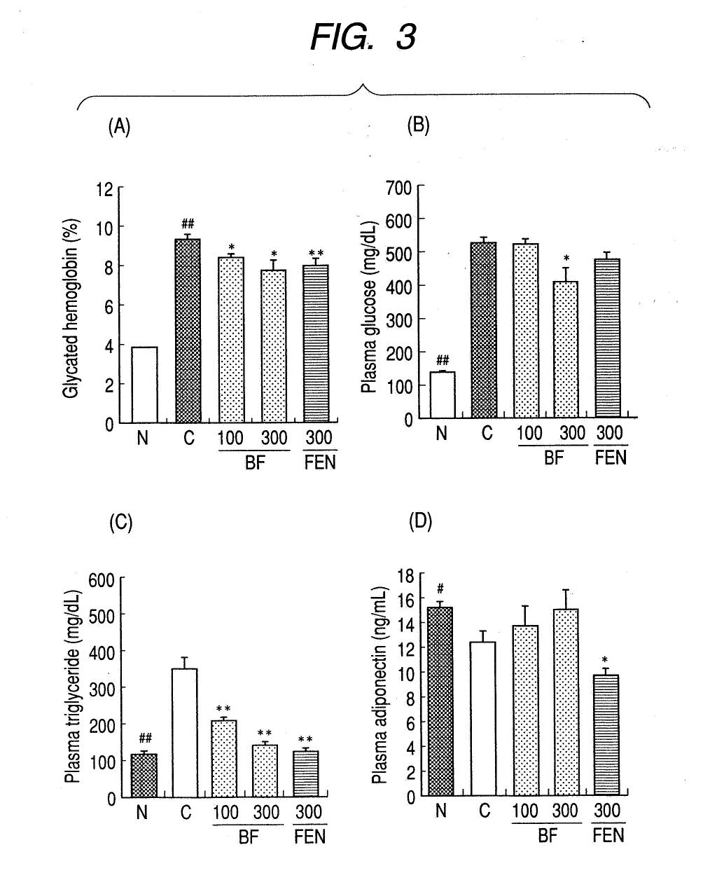

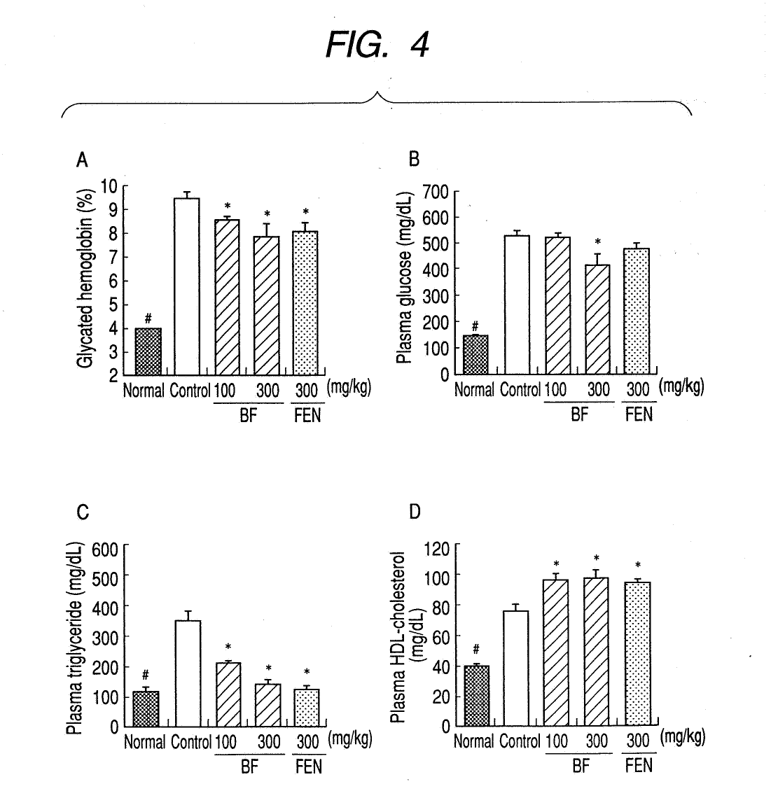

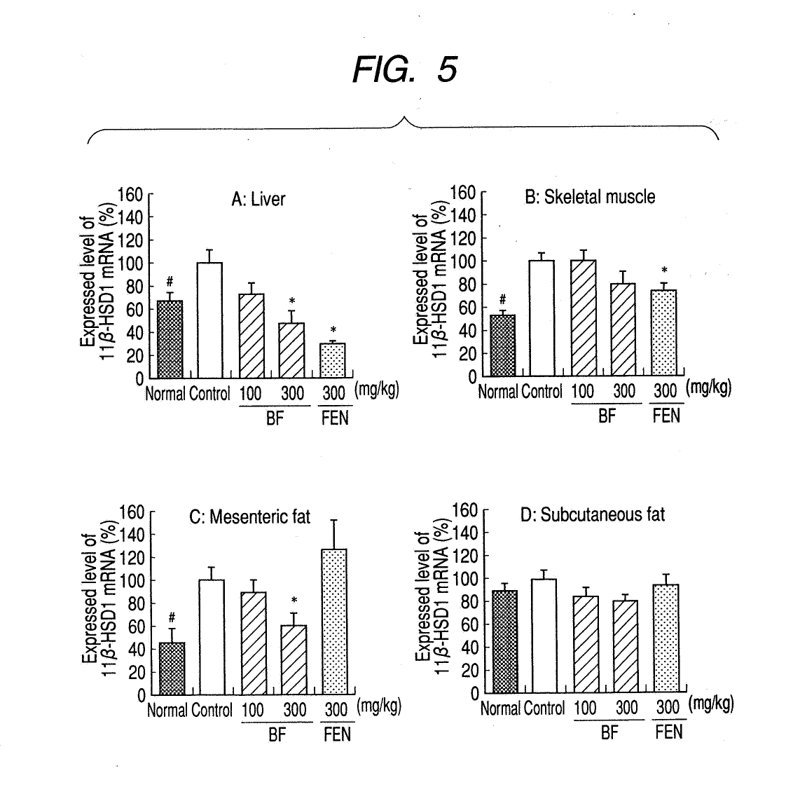

A 1% methyl cellulose solution (control group), 100 mg/kg or 300 mg/kg of bezafibrate or 300 mg/kg of fenofibrate was orally administered to the db/db mice repeatedly once a day. After 8 weeks of the administration, blood was drawn from the caudal vein to measure blood glycated hemoglobin value, plasma glucose concentration, plasma triglyceride concentration and plasma adiponectin concentration. The results are shown in FIG. 3.

In comparison with db/db control mice, repeated administration of bezafibrate significantly reduced the blood glycated hemoglobin value, plasma glucose concentration and plasma triglyceride concentration of after 8 weeks. On the other hand, in comparison with the control group, repeated administration of fenofibrate significantly reduced the plasma triglyceride concentration after 8 weeks.

As shown in Example 1 to Example 3, bezafibrate and fenofibrate improved diabetes and hyperlipemia of db/db mice.

### Example 4

A 1% methyl cellulose solution (db/db control mice), 100 mg/kg or 300 mg/kg of bezafibrate or 300 mg/kg of fenofibrate was orally administered repeatedly once a day for 8 weeks to type 2 diabetic mice, db/db mice, and normal. At 8 weeks after the commencement of the repeated administration, blood was drawn from the caudal vein to measure blood glycated hemoglobin value, plasma glucose concentration, triglyceride concentration and HDL-cholesterol concentration.

The measured results are shown in FIG. 4.

In comparison with the db/db control mice, both of bezafibrate and fenofibrate significantly reduced the glycated hemoglobin value and plasma triglyceride concentration, and plasma increased HDL-cholesterol concentration after 8 weeks. In addition, bezafibrate significantly reduced the plasma glucose concentration of after 8 weeks, in comparison with the db/db control mice.

Accordingly, bezafibrate and fenofibrate can improve diabetes and hyperlipemia of db/db mice and alleviate the risk for arteriosclerotic diseases by the action to increase HDL-cholesterol concentration.

### Example 5

After 8 weeks of the commencement of the administration, each animal was anesthetized by the intraperitoneal injection of 20% chloral hydrate (manufactured by Wako Pure Chemical Industries) to remove liver, skeletal muscle, mesenteric adipose (visceral fat) tissue and subcutaneous adipose tissue. Total RNA was extracted from each of the thus removed tissues using an RNA extraction reagent ISOGEN (manufactured by Nippon Gene), and the total RNA was further purified using RNeasy Micro Kit (manufactured by Qiagen). Using the thus purified total RNA of liver, skeletal muscle, mesenteric adipose tissue or subcutaneous adipose tissue as the template, expression level of 11β-HSD1 mRNA in each tissue was determined by carrying out real time quantitative RT-PCR. GeneAmp 5700 Sequence Detection System (manufactured by Applied Biosystems) was used in the reaction.

The results are shown in FIG. 5.

In comparison with db/db control mice, bezafibrate and fenofibrate significantly suppressed expression of 11β-HSD1 in the liver.

In comparison with db/db control mice, fenofibrate significantly suppressed expression of 11β-HSD1 in the skeletal muscle.

In comparison with db/db control mice, bezafibrate significantly suppressed expression of 11β-HSD1 in the mesenteric fat. Accordingly, bezafibrate can be used in the prevention or treatment of obesity with visceral fat accumulation by improving abnormal activation of the glucocorticoid action in visceral fat.

In addition, both of bezafibrate and fenofibrate did not show the effect to suppress expression of 11β-HSD1 in subcutaneous fat.

Based on the above, bezafibrate can be used as an agent for preventing or treating metabolic syndrome.

While the invention has been described in detail and with reference to specific embodiments thereof, it will be apparent to one skilled in the art that various changes and modifications can be made therein without departing from the scope thereof.

This application is based on Japanese patent applications No. 2005-303264 filed Oct. 18, 2005 and No. 2006-010882 filed Jan. 19, 2006, the entire contents thereof being hereby incorporated by reference.

What is claimed is:

1. A method for preventing or treating metabolic syndrome, which comprises administering bezafibrate.

2. A method for preventing or treating obesity, which comprises administering bezafibrate.

3. The prophylactic or therapeutic agent described in claim 2, wherein the obesity is obesity with visceral fat accumulation.

4. A method for inhibiting expression of 11β-hydroxysteroid dehydrogenase type 1, which comprises administering bezafibrate.
